(19) United States
(12) Patent Application Publication
(10) Pub. No.: US 2024/0082474 A1
(43) Pub. Date: Mar. 14, 2024

(54) Devices and methods for fistula-free hemodialysis

(71) Applicant: Sorin Grunwald, Pantin (FR)

(72) Inventor: Sorin Grunwald, Pantin (FR)

(21) Appl. No.: 18/278,024

(22) PCT Filed: Mar. 25, 2022
(86) PCT No.: PCT/EP2022/057987

§ 371 Date: Aug. 21, 2023

Related U.S. Application Data
(62) Division of application No. PCT/EP2022/057987, filed on Mar. 25, 2022.
(60) Provisional application No. 63/167,771, filed on Mar. 30, 2021.
(60) Provisional application No. 63/168,276, filed on Mar. 31, 2021.
(60) Provisional application No. 63/168,277, filed on Mar. 31, 2021.
(60) Provisional application No. 63/222,062, filed on Jul. 15, 2021.

(51) Int. Cl. A61M 1/36; A61M 39/02

(52) U.S. Cl. CPC A61M 1/3653; A61M 39/0247

(57) ABSTRACT

Devices and methods for fistula-free vascular access for hemodialysis are disclosed. In exemplary embodiments, devices are provided comprising a transcutaneous access port (200) for needlelessly connecting a patient to a dialyzer, the port having different inner lumen profiles for arterial or venous blood flow and further comprising an intracorporeal conduit providing a fluid path between the transcutaneous port and a blood vessel, wherein the intracorporeal conduit is attached to a device anchor (400) having distal and proximal self-expandable segments for anastomosing the intracorporeal conduit to a blood vessel. An exemplary fistula-free hemodialysis method (100) provided herein consists of percutaneously deploying a first device in an artery and a second device in a vein and independently connecting each of the devices to the arterial and venous lines of a dialyzer.

### REFERENCES CITED

### Cross Reference to Related Applications

This application is a division of PCT Application No. PCT/EP2022/057987 filed on 25 Mar. 2022, International Publication Number WO 2022/207505 A2 and claims priorities to:
1. U.S. Provisional Patent Application Serial No. U.S. 63/167,771 filed on Mar. 30, 2021 (Needleless transcutaneous port)
2. U.S. Provisional Patent Application Serial No. U.S. 63/168,276, filed on Mar. 31, 2021 (Anastomosis device and method for percutaneous delivery thereof)
3. U.S. Provisional Patent Application Serial No. U.S. 63/168,277, filed on Mar. 31, 2021 (Devices and methods for Hemodialysis)
4. U.S. Provisional Patent Application Serial No. U.S. 63/222,062, filed on Jul. 15, 2021 (Devices and methods for sutureless anastomosis)
the disclosures of which are incorporated herein by reference in their entirety.

U.S. PATENT DOCUMENTS

| | | | |
|---|---|---|---|
| 3,628,813 | December 1971 | Lee | Fluid connecting device |
| 4,007,743 | February 1977 | Blake | Opening mechanism for umbrella-like intravascular shunt |
| | | | defect closure device |
| 4,352,358 | October 1982 | Angelchik | Apparatus for effecting anastomotic procedures |
| 4,353,368 | October 1982 | Slovak et al. | Device for hemodialysis |
| 4,569,675 | February 1986 | Prosl et al. | Transcutaneous infusion system |
| 4,892,518 | January 1990 | Cupp et al. | Hemodialysis |
| 4,968,422 | November 1990 | Runge et al. | Pulsatile flow hemodialysis |
| 5,035,702 | July 1991 | Taheri | Method and apparatus for providing an anastomosis |
| 5,342,393 | August 1994 | Stack | Method and device for vascular repair |
| 5,456,712 | October 1995 | Maginot | Graft and stent assembly |
| 5,562,617 | October 1996 | Finch, Jr. et al. | Implantable vascular device |
| 5,755,775 | May 1998 | Trerotola et al. | Percutaneous stent-graft and method for delivery thereof |
| 5,755,778 | May 1998 | Kleshinski | Anastomosis device |
| 5,861,003 | January 1999 | Latson et al. | Apparatus and method for occluding a defect or aperture |
| | | | within body surface |
| 5,911,706 | June 1999 | Estabrook et al. | Device for subcutaneous accessibility |
| 5,944,688 | August 1999 | Lois | Implantable hemodialysis access port assembly |
| 6,036,702 | March 2000 | Bachinski et al. | Medical grafting connectors and fasteners |
| 6,231,541 | May 2001 | Kawamura | No-needle blood access device for hemodialysis and no- |
| | | | needle connecting cannula assembly |
| 6,312,446 | November 2001 | Huebsch et al. | Apparatus and method for closing a septal defect |
| 6,402,764 | June 2002 | Henricksen et al. | Everter and threadthrough system for attaching graft |
| | | | vessel to anastomosis device |
| 6,428,550 | August 2002 | Vargas et al. | Sutureless closure and deployment system for connecting |
| | | | blood vessels |
| 6,648,901 | November 2003 | Fleischman et al. | Anastomosis system |
| 6,719,781 | April 2004 | Kim | Catheter apparatus having an improved shape-memory alloy |
| | | | cuff and inflatable on-demand balloon for creating a bypass |
| | | | graft in-vivo |
| 6,776,785 | August 2004 | Yencho et al. | Implantable superelastic anastomosis device |
| 7,585,306 | September 2009 | Abbott et al. | Anastomosis device, tools and methods of using |
| 7,762,977 | July 2010 | Porter et al. | Device and methods for vascular access |
| 8,277,465 | October 2012 | Beane et al. | Apparatus and method for connecting a conduit to a hollow |
| | | | vessel |
| 8,747,344 | June 2014 | Khan | Hybrid arteriovenous shunt |
| 8,845,573 B2 | September 2014 | Häusler et al. | Implantable access for removal and/or return of fluids; |
| | | | reference cited in the written opinion of the International |
| | | | Searching Authority [19] |
| 8,900,288 | December 2014 | Chobotov et al. | Advanced endovascular graft delivery system and method |
| | | | of treatment |
| 8,926,591 | January 2015 | Schutz et al. | Implantable vascular access |
| 9,247,930 | February 2016 | Coleman et al. | Device and methods for occluding or promoting fluid flow |
| 9,597,443 | March 2017 | Yevzlin et al. | Anastomotic connector |
| 9,820,743 | November 2017 | Asfora et al. | Sutureless vascular anastomosis connection |
| 10,702,688 | July 2020 | Mason et al. | Vascular access device |
| 10,166,321 | January 2019 | Casiello et al. | High-flow port and infusion needle systems |
| 10,751,056 | August 2020 | Porter | Methods and apparatus for percutaneous bypass graft |
| 10,773,010 | September 2020 | Phillips et al. | Subcutaneous vascular access ports and related systems and |
| | | | methods (needle guiding devices) |
| 10,835,366 | November 2020 | Donadio, III et al. | Arterial and venous anchor devices forming an anastomotic |
| | | | connector and system for delivery |
| 10,835,663 | November 2020 | Peh et al. | Subcutaneous implantable device for guiding a vascular |
| | | | access member and method for vascular access |
| US 2005/143758 | A1 | (ABBOT RYAN | 30 Jun. 2005, reference cited in the written opinion of |
| | | [US] ET AL) | the International Searching Authority [19]. |
| US 2014/100510 | A1 | (YEVZLIN ALEXANDER S | 10 Apr. 2014, reference cited in the written |
| | | [US] ET AL) | opinion of the International Searching Authority [19] |
| US 2020/114060 | A1 | (VARTANIAN SHANT | 16 Apr. 2020, reference cited in the written opinion of |
| | | [US] | the International Searching Authority [19] |

### FOREIGN PATENT DOCUMENTS

| | | | | |
|---|---|---|---|---|
| WO | 97/42878 | November 1997 | Kotula et al. | Percutaneous catheter directed |
| | | | | intravascular occlusion devices |
| WO | 2020/004672 A1 | 2 Jan. 2020 | reference cited in the | |
| | (KK ADVANCE [JP]) | (2020 Jan. 2) | written opinion of the International | |
| | | | Searching Authority [19] | |

### OTHER PUBLICATIONS

1. Agarwal A. K. et al., Innovations in vascular access for hemodialysis, Kidney International (2019) 95, 1053-1063
2. Allon M., Vascular Access for Hemodialysis Patients—New Data Should Guide Decision Making, American Society of Nephrology, CJASN Vol 14 June, 2019
3. Canaud B. et al., Vascular Access Management for Haemodialysis: A Value-Based Approach from NephroCare Experience, IntechOpen 2019
4. Ilie V. et al., Sutureless Microvascular Anastomosis: Literature Review, International Microsurgery Journal, 2019; 3(2):1
5. Mallios A. et al., Early results of percutaneous arteriovenous fistula creation with the Ellipsys Vascular Access System, Journal of Vascular Surgery, 2018
6. Al-Jaishi A. A. et al., Complications of the Arteriovenous Fistula: A Systematic Review, J Am Soc Nephrol 28: 1839-1850, 2017
7. Al-Balas A. et al., The Clinical and Economic Effect of Vascular Access Selection in Patients Initiating Hemodialysis with a Catheter, J Am Soc Nephrol 28: 3679-3687, 2017
8. Al Shakarchi J. et al., Early cannulation grafts for hemodialysis: a systematic review, J Vasc Access 2015; 16(6): 493-497
9. Parisotto, M. T., et al., Cannulation technique influences arteriovenous fistula and graft survival, Kidney International (2014) 86, 790-797
10. Ebner A. et al., Minimally Invasive Sutureless Anastomosis of an AV Hemodialysis Graft, Endovascular Today, November 2014
11. Stolic R, Most Important Chronic Complications of Arteriovenous Fistulas for Hemodialysis, Medical Principles and Practice 2013; 22:220-228
12. Vachharajani T. J., Atlas of Dialysis Vascular Access, Wake Forest University, School of Medicine, 2010
13. Hadaway L. et al., Needleless Connectors: A Primer on Terminology, Journal of Infusion Nursing, January 2010
14. Misra M., The basics of hemodialysis equipment, Hemodialysis International 2005; 9:3-36
15. Twardowski Z. J. et al., Blood Flow, Negative Pressure, and Hemolysis During Hemodialysis, Home Hemodial Int, Vol 3, 45-50. 1999
16. Foran R. F., Quinton-Scribner Cannulas for Hemodialysis—Review of Four Years' Experience, The Western Journal of Medicine, March 1970, 112-3
17. Brescia M. J. et al., Chronic hemodialysis using venipuncture and a surgically created arterio-venous fistula, N Engl J Med. 1966; 275:1089-1092
18. Clark P. B., Routine Use of Scribner Shunt for Haemodialysis, British Medical Journal, May 1966: 1200-1202
19. PCT, INTERNATIONAL SEARCH REPORT AND WRITTEN OPINION OF THE INTERNATIONAL SEARCHING AUTHORITHY mailed on 17 November, 2022 for International Application No. PCT/EP2022/057987, International Publication Number WO 2022/207505 A2.

### TECHNICAL FIELD

The present invention relates generally to devices and methods that can be used for vascular access for hemodialysis. More specifically, the present invention relates to devices and methods that can be delivered percutaneously and suturelessly and that do not require an arteriovenous fistula, an arteriovenous graft, or a central venous catheter for vascular access (fistula-free hemodialysis). Further, the present invention relates to devices for improved and needleless connection of a patient to a dialyzer.

### BACKGROUND

Clinical Background

Worldwide, hemodialysis (HD) remains the prevalent dialysis modality for more than 2 million patients with end-stage renal disease. The health care expenditure to treat end-stage renal disease has increased to approximately $34 billion in 2015 in the United States alone. A significant portion of this expense is related to the establishment and maintenance of vascular access (VA) for HD [1].

The 2006 Kidney Disease Outcomes Quality Initiative (KDOQI) VA guidelines state that “options for fistula placement should be considered first.” However, more recent publications have reported 30%-60% arteriovenous fistula (AVF) nonmaturation rates for AVFs that cannot be used or require assisted maturation [2]. Although the AVF is currently considered the ideal model of VA for HD, AVFs are the source of complications, the most important being lymphedema, infection, aneurysm, stenosis, congestive heart failure, steal syndrome, hand ischemia and thrombosis. Fistula complications are associated with morbidity, mortality, and a high economic burden [6]. Several studies indicate that about 30% of HD hospitalizations are caused by VA construction and complications of vascular access [11]. Endovascular creation has simplified the creation of AVFs using either radiofrequency (everlinQ, TVA Medical, Austin, TX, USA) or thermal resistance (Ellipsys, Avenu Medical, San Juan Capistrano, CA, USA) arteriovenous anastomosis devices [1], [5]. However, many of clinical issues and complications related to AVFs, as mentioned above, have remained unaddressed.

If a patient is not a suitable candidate for AVF, arteriovenous grafts (AVG) is the second vascular access option for HD. Compared to the AVF, the AVG has better mechanical strength, earlier use, and primary failure rates but also increased risk of developing graft stenosis, a fivefold increase in infection risk, a poorer long-term patency, higher levels of complications and requires more interventions than AVF [2]. Central venous catheters (CVC) are also used in specific cases for starting hemodialysis because they provide immediate vascular access. However, prolonged CVC use results in high risk of CVC-related bloodstream infections. CVCs are also associated with a higher risk of central vein stenosis and decreased survival rates [2].

### RELATED ART

The creation of an AVF for long-term HD in 1966 by Cimino and Brescia was the first major innovation after the Scribner shunt first described in 1961 and was followed by the development of arteriovenous grafts and hemodialysis catheters. To date, the innovations in vascular access for hemodialysis have focused on arteriovenous fistulas, arteriovenous grafts, central venous catheters and the process of healthcare. Innovations related to arteriovenous fistulas have focused on: a) endovascular creation; b) (sutureless) anastomotic devices; c) maturation facilitators; and d) superificialization. Innovations related to arteriovenous grafts have focused on: a) early access AVG; b) heparin-bonded AVG; c) hybrid catheter graft; d) hybrid graft-stent; and e) tissue bioengineered vessels. Innovations related to central venous catheters have focused on: a) catheter tip design; b) catheter coating; and c) catheter lock solutions. Innovations related to the process of healthcare have focused on: a) patient-centric care; b) value versus volume-based care; c) preemptive versus reactive care; and d) evidence-based care to reduce health care-associated infections [1].

Foran and Clark describe the use of a device, the Scribner shunt, that provides separate arterial and venous access for HD. Some of the most important benefits of the Scribner shunt are: a) the ease-of-use for repetitive HD access compared to new cannulations; and b) the ability to use the shunt for HD access immediately after implantation. Some of the limitations of the Scribner shunt were: a) the fact that the device was an extracorporeal device which lead to a higher risk of infection, in particular at the points of insertion; b) the arterial and venous indwelling segments of the devices caused thrombosis and stenosis; c) the high complexity of the surgical insertion and fixation of the cannula tip, particularly in the artery; and d) the material of the device caused clotting and progressive deterioration of the cannula function.

In U.S. Pat. No. 6,231,541 Kawamura describes a no-needle blood access device for HD comprising an artificial conduit whose opposite ends are anastomosed to a target artery or vein. The device contains a transcutaneous component that allow for connecting the subcutaneous conduit to an extracorporeal HD machine. Some of the limitations of Kawamura's disclosure are: the disclosed device requires a fistula or graft to connect a vein to an artery, the fistula/graft is created through open surgery and sutured to the target vessels; there is only one skin access point for both the arterial and venous lines for hemodialysis limiting the choice of target blood vessels and access points; the use of disclosed shutters to open and close conduits leads to blood loss when connecting a dialyzer; the long cannulas inserted in the blood stream increase the risk of infections; and once implanted, the device cannot be replaced if it fails in time.

In U.S. Pat. No. 6,719,781 Kim describes a catheter apparatus, an improved introducer system, and a methodology for creating a bypass using a prepared shape-memory alloy cuff and a graft segment in tandem as a shunt. Some of the limitations of Kim's disclosure are: the cuff is fixed to the graft by the surgeon through suturing during the procedure and the fixing means can cause tissue damage; the cuff expands based on thermal properties and needs cooling until deployment; the disclosed device can only be used for large arteries (aorta) at one end of the graft while the other end of the graft is connected surgically with sutures by the surgeon in a standard surgical procedure; and, in general, grafts cannot be connected to a dialyzer considering the conduit sizes and fluid pressures required by the hemodialysis process.

In U.S. Pat. No. 6,776,785 Yencho et al. describe a one-piece anastomosis device formed of a superelastic or pseudoelastic material which self-deforms or self-deploys from an insertion configuration to a tissue holding configuration. The self-deploying anastomosis device does not rely on a temperature transformation to achieve deployment. Some of the limitations of Yencho's disclosure are: the disclosed device can only be used to connect a tissue graft and cannot be used to connect a catheter or other semi-rigid conduit and, thus, cannot be used for hemodialysis; the risk of damaging the blood vessel is high during the deployment procedure due to the lack of maneuverability, and the device cannot be placed without direct visualization of the target vessel.

In U.S. Pat. No. 7,585,306 Abbott et al. describe anastomosis devices, tools and methods of performing sutureless anastomosis. Anastomosis devices are provided for fixing a first conduit to a second conduit in an anastomosis, where the conduits are joined by interfacing their inner walls together. Some of the limitations of Abbott's disclosure are: the devices can be used only for large blood vessels, e.g. aorta, because of the reverted graft attaching mechanism; the device cannot be connected to a catheter or other semi-rigid conduit and, thus, cannot be used for hemodialysis;

In U.S. Pat. No. 9,247,930 Coleman et al. describe devices and sutureless percutaneous methods for occluding or promoting fluid flow through openings. In an exemplary embodiment an occlusion device is provided having an outer elongated tubular body that is configured to expand and form proximal and distal wings proximate to opposed ends of the opening. The device can include a component to occlude flow through the tubular body and thus through the opening. Some of the limitations of Coleman's disclosure are: the disclosed device cannot be used to promote flow in small vessels because of the inherent wall thickness of the disclosed device structure. A device with a small outer diameter and an even smaller inner diameter cannot ensure the fluid debits required for hemodialysis, i.e., 300-600 ml/min; in addition, when promoting flow, certain disclosed device components must reside inside the target blood vessel obstructing flow in said blood vessel. Thus, the device can only be used for bypass procedure where the blood flow is already obstructed in the target vessel. Moreover, the disclosed device is made of a solid stainless steel structure that is too heavy to attach to small vein walls that would collapse under the device weight.

In U.S. Pat. No. 9,597,443 Yevzlin et al. describe an anastomotic connector comprising a tubular access port and an anchor with a plurality of fingers to be extended in a blood vessel connected to the tubular access port. Some of the limitations of Yevzlin's disclosure are: the disclosure does not allow for percutaneous deployment of a catheter pre-connected to the disclosed anastomotic connector thus limiting the choice of anastomosis locations to those directly accessible by open surgery; the proximal end of the anastomotic connector that resides outside the blood vessel is fixed in shape and perpendicular to the longitudinal axis of the blood vessel, said characteristics further limiting the maneuverability of the device and the ability to connect it to smaller blood vessels.

Clinical Need

What is needed are improved devices and methods for vascular access for hemodialysis that reduce the risks associated with establishing and maintaining vascular access, decrease time to first use, increase long-term patency, minimize complications, simplify the hemodialysis procedure, and reduce the burden on patients and on the healthcare system.

### SUMMARY

Devices and methods for fistula-free vascular access for hemodialysis are disclosed herein. In exemplary embodiments, devices are provided including a transcutaneous access port for needlelessly connecting a patient to a dialyzer, an intracorporeal conduit providing a fluid path between said transcutaneous port and a blood vessel, and a device anchor, said device anchor permitting the sutureless anastomosis of said intracorporeal conduit to said blood vessel. In exemplary embodiments, methods are provided for creating fistula-free vascular access for hemodialysis and performing hemodialysis procedures without needle punctures.

In one exemplary embodiment, an implantable transcutaneous access port disclosed herein comprises a port body with an inner lumen and a subcutaneous segment attachable to an intracorporeal conduit, wherein the longitudinal axis of said subcutaneous segment and the longitudinal axis of said port body are at a variable angle with respect to each other in order to adapt to the anatomy of the access and implant locations and wherein said inner lumen has different profiles such as to provide variable blood flow characteristics adapted to either arterial or venous blood flow and, wherein the port body can be connected to an extracorporeal connector and to a dialyzer.

In one exemplary embodiment, an intracorporeal conduit disclosed herein comprises a distal more rigid segment and a proximal less rigid segment, said proximal less rigid segment being attached to a transcutaneous access port and said distal more rigid segment being fixedly attached to and within a tubular device anchor, wherein said distal more rigid segment extends through the inner lumen of said device anchor such that the distal end of said segment and the distal end of said device anchor are flush, and wherein the outer diameter of said distal more rigid segment of said conduit is equal to or slightly smaller than the inner diameter of said device anchor.

In one exemplary embodiment, a device anchor disclosed herein for suturelessly anastomosing the distal end of an intracorporeal conduit to a blood vessel or graft comprises a tubular body with a pre-shaped self-expandable proximal segment, a pre-shaped self-expandable waist segment, and a pre-shaped self-expandable distal segment, wherein said proximal segment self-expands outside a wall of said blood vessel or graft, the length of the said waist segment is approximately the size of the thickness of said wall and said distal segment self-expands inside said blood vessel or graft or inside a tubular device implanted in the blood vessel or graft as to, together with the self-expanded proximal segment, apply sufficient pressure on the vessel wall in order to keep said device steadily attached to said wall without penetrating it, wherein the proximal end of said device anchor slides freely over said distal more rigid segment of said intracorporeal conduit during compression of said device anchor and during self-expansion of said device anchor from a compressed configuration to its pre-shaped expanded configuration, and wherein said device anchor comprises, at its proximal end, an attaching and detaching mechanism for the attachment and detachment of a delivery element for the percutaneous deployment of said device.

In one exemplary embodiment, a fistula-free hemodialysis method disclosed herein consists of percutaneously deploying a first said intracorporeal conduit and anastomosing it to an artery for creating arterial access and a second said intracorporeal conduit and anastomosing it to a vein for creating venous access and independently connecting each of said devices respectively to the arterial and venous lines of said dialyzer using said transcutaneous ports.

The contributions of the present invention in addressing current hemodialysis needs with respect to prior art are manyfold. The devices disclosed in the present invention can be delivered percutaneously and without employing sutures and provide independent access to arteries and veins. The methods disclosed herein allow for the creation of fistula-free vascular access for hemodialysis without the need for an arteriovenous fistula, arteriovenous graft or central venous catheter. Thus, the risks and issues associated with the creation and maintenance of vascular access for hemodialysis are significantly reduced. Moreover, the devices disclosed herein provide access for required hemodialysis debits of 300-600 ml/min to and from blood vessels as small as 2-3 mm in diameter, thus increasing the number of choices for vascular access points in a patient's vasculature. Further, the devices and methods disclosed herein allow for a needleless connection between a patient and a dialyzer, thus simplifying and decreasing the burden of the hemodialysis procedure in hospitals and homecare settings.

### BRIEF DESCRIPTION OF THE DRAWINGS

FIG. 1 illustrates an example embodiment of a method of use of the present invention.

FIG. 2 illustrates an example embodiment of a transcutaneous access port according to the present invention.

FIG. 3 illustrates an example embodiment of a device anchor according to the present invention.

FIG. 4 illustrates an example embodiment of a device anchor connected to the distal end of an intracorporeal conduit according to the present invention.

FIG. 5 illustrates an example embodiment of a device anchor with a variable angle between said anchor body and its expandable segments.

FIG. 6 illustrates an example embodiment of an intracorporeal conduit deployed and attached to a blood vessel or graft using a device anchor according to the present invention.

FIG. 7 illustrates a superimposed view of an expanded and non-expanded example embodiment of a device anchor attached to a conduit according to the present invention.

FIG. 8A illustrates a 3D view of an example embodiment of an expanded (pre-shaped or preprogrammed) device anchor according to the present invention.

FIG. 8B illustrates 3D view of an example embodiment of a non-expanded device anchor according to the present invention.

FIG. 9A illustrates a circular cross-sectional view of an expanded distal end of an example embodiment of a device anchor according to the present invention.

FIG. 9B illustrates an elliptical cross-sectional view of an expanded distal end of another example embodiment of a device anchor according to the present invention.

FIG. 10 illustrates a 3D view of an example embodiment of a laser cut strut pattern for a device anchor in a non-expanded configuration according to the present invention.

FIG. 11 illustrates a 3D view of an example embodiment of a device anchor in an expanded configuration anastomosed to a blood vessel or graft according to the present invention.

FIG. 12 illustrates a lateral view of another non-expanded example embodiment laser cut strut pattern of a device anchor suited for deployment at an angle with respect to a target vessel according to the present invention.

FIG. 13 illustrates a frontal view of another non-expanded example embodiment laser cut strut pattern of a device anchor suited for deployment at an angle with respect to the target vessel according to the present invention.

FIG. 14 illustrates a superimposed view of an expanded (pre-shaped or preprogrammed) and non-expanded example embodiment laser cut strut pattern of a device anchor suited for deployment at an angle with respect to the target vessel according to the present invention.

FIG. 15 illustrates an example embodiment of the deployment of a device anchor at an angle with respect to a target vessel according to the present invention.

FIG. 16A illustrates a 3D view of another example embodiment laser cut strut pattern of a device anchor in a non-expanded configuration that minimizes device weight and contact surface with the target conduit according to the present invention.

FIG. 16B illustrates a 3D superimposed view of another example embodiment of an expanded (pre-shaped or preprogrammed) and non-expanded device anchor according to the present invention.

FIG. 17A illustrates a 3D view of another example embodiment of an expanded (pre-shaped or preprogrammed) device anchor attached to the distal segment of an intracorporeal conduit according to the present invention.

FIG. 17B illustrates a cross-sectional view of the device illustrated at FIG. 17A.

FIG. 18A illustrates a 3D view of an example embodiment laser cut strut pattern of a device anchor with a female-side bayonet attaching mechanism in a non-expanded configuration according to the present invention.

FIG. 18B illustrates a 3D view of an example embodiment of an expanded (pre-shaped or preprogrammed) device anchor with a female-side bayonet attaching mechanism, said anchor being attached to a distal segment of an intracorporeal conduit according to the present invention.

FIG. 19A illustrates a lateral view of an example embodiment of a delivery element with a male-side bayonet attaching mechanism according to the present invention.

FIG. 19B illustrates a longitudinal sectional view of an example embodiment of a delivery element with a male-side bayonet attaching mechanism according to the present invention.

FIG. 19C illustrates a cross-sectional view of an example embodiment of a delivery element with a male-side bayonet attaching mechanism according to the present invention.

FIG. 20 illustrates an example embodiment of a method of loading a device anchor and intracorporeal conduit into a loader using a delivery element according to the present invention.

FIG. 21 illustrates an example embodiment of a device deployment kit for percutaneous and sutureless deployment of an intracorporeal conduit and device anchor according to the present invention.

FIG. 22 illustrates an example embodiment of a method for percutaneous and sutureless attachment of an intracorporeal conduit to a blood vessel or graft using a device anchor and a device deployment kit according to the present invention.

FIG. 23 illustrates an example embodiment of a device and method for needlelessly connecting a patient to a dialyzer or to another external device according to the present invention.

FIG. 24 illustrates another example embodiment of a transcutaneous access port placed subcutaneously according to the present invention.

FIG. 25 illustrates another example embodiment of a transcutaneous access port placed subcutaneously and comprising an adaptor for several predefined needle access points (buttonholes) according to the present invention.

### DETAILED DESCRIPTION

While the invention is described herein in detail with reference to the preferred embodiments thereof, it will be apparent to one skilled in the art that various changes and modifications can be made and equivalents employed, without departing from the present invention. Further, a person skilled in the art will appreciate that the devices disclosed herein can be adapted for use in any of the methods disclosed herein, and likewise, the methods disclosed herein can be adapted for use in conjunction with any of the devices disclosed herein. In order to streamline the disclosure of the present invention, not all elements of one embodiment are described in detail but only those specifically targeted in that particular embodiment. For example, an embodiment may be illustrated comprising a valve and another without a valve. It will be apparent to one skilled in the art that such elements, like the above mentioned valve, may be or not be present in all configurations and drawings without affecting the objective, spirit, and scope of the present invention. In addition, modifications may be made to adapt a particular situation, material, shape, dimension, processor, or method to the objective, spirit, and scope of the present invention. All such modifications are intended to be within the scope of the claims appended hereto. In the present disclosure, like-numbered components of embodiments generally have similar features, and thus within a particular embodiment each feature of like-numbered component is not necessarily fully elaborated upon in the description of the particular embodiment.

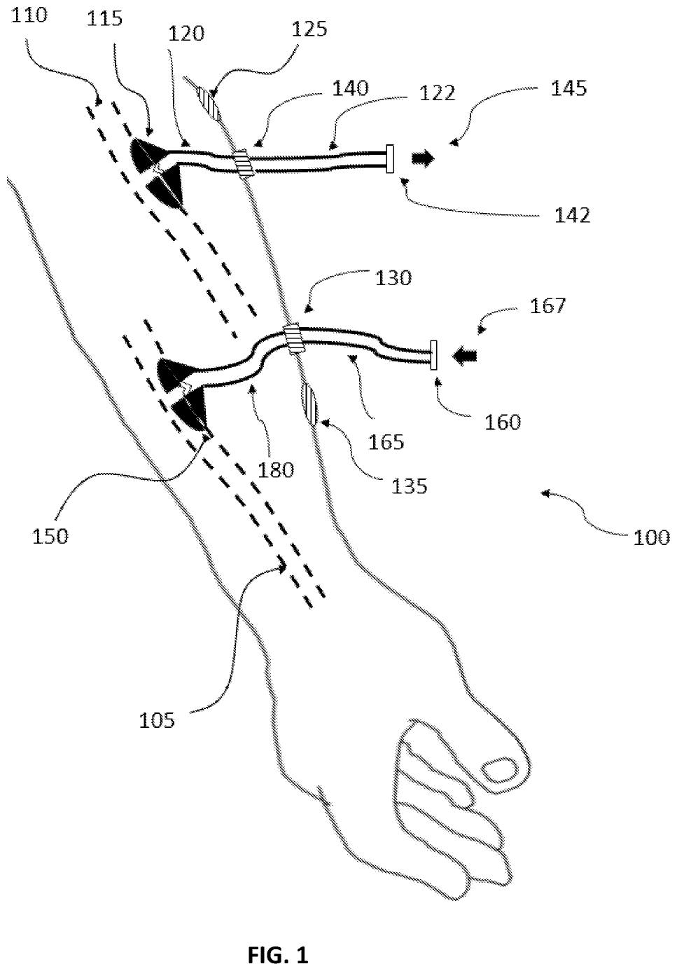

FIG. 1 illustrates an exemplary context of use 100 of the present invention. In order to create vascular access for hemodialysis, a first intracorporeal conduit 120 is deployed percutaneously through a skin area 125 and attached to the wall of a conduit in the body, e.g., to the wall of a blood vessel or graft 110 using the device anchor 115 as described further herein. In one embodiment of the present invention, blood vessel 110 is an artery. In one embodiment of the present invention, the conduit 120 is attached to transcutaneous access port 140 in order to facilitate the connection of conduit 120 to an external device using conduit 122 and connector 142. In one embodiment of the present invention, transcutaneous port 140 is attached to the patient's skin. In one embodiment of the present invention, conduit 122 can be attached to and detached from transcutaneous access port 140. In one embodiment of the present invention, connector 142 is attached to the arterial line of a hemodialysis machine (dialyzer) and blood can be drawn (145) from artery 110 into the dialyzer during the hemodialysis procedure.

A second intracorporeal conduit 180 is deployed percutaneously through a skin area 135 and attached to the wall of a conduit in the body, e.g., to the wall of a blood vessel or graft 105 using the device anchor 150 as described further herein. In one embodiment of the present invention blood vessel 105 is a vein. In one embodiment of the present invention, conduit 180 is attached to transcutaneous port 130 in order to facilitate the connection of conduit 180 to an external device using conduit 165 and connector 160. In one embodiment of the present invention, transcutaneous access port 130 is attached to the patient's skin. In one embodiment of the present invention, conduit 165 can be attached to and detached from transcutaneous access port 130. In one embodiment of the present invention, connector 160 is attached to the venous line of a hemodialysis machine (dialyzer) and blood can be infused (returned) (167) into vein 105 from the dialyzer during the hemodialysis procedure.

In another embodiment of the present invention, a first and a second intracorporeal conduits can be attached (anastomosed) at different locations on the same conduit in the body, e.g., on the same vein or a graft.

In another embodiment of the present invention, only one intracorporeal conduit and transcutaneous port is deployed, e.g., to a vein or graft for single needle hemodialysis.

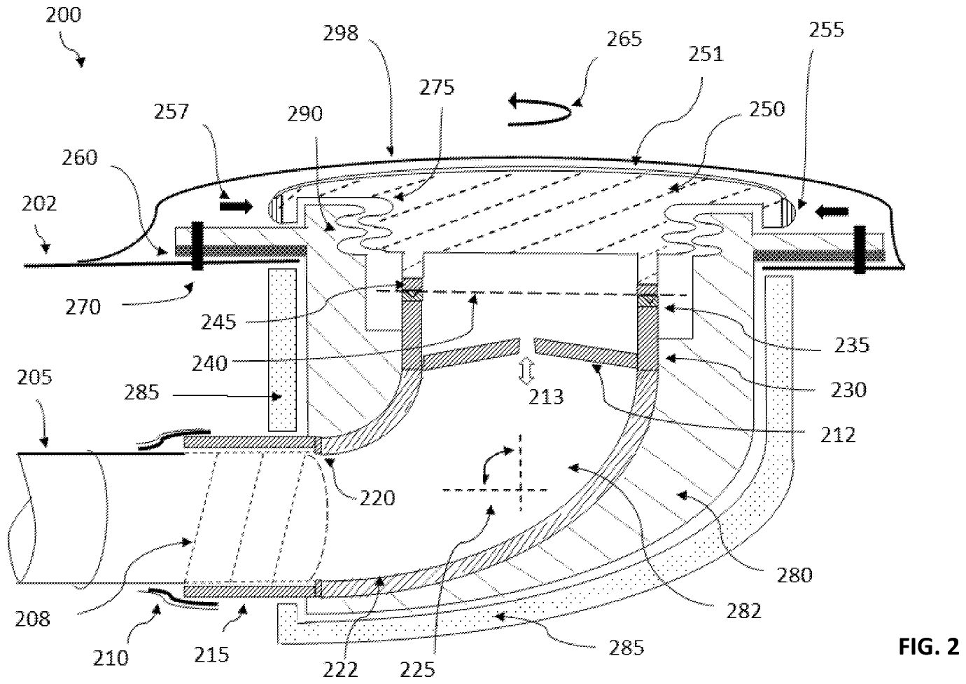

FIG. 2 illustrates an example embodiment of a transcutaneous access port 200 according to the present invention. Said port allows for repetitive needles access to an intracorporeal conduit 205 in a patient's body through the patient's skin 202. A subcutaneous segment 215 of port 200 can be connected to the proximal end of said conduit 205 as described herein. The distal end of conduit 205 can be indwelling in a patient's blood vessel or anastomosed to another conduit of the human body, e.g., to a blood vessel or to an arteriovenous graft.

According to one embodiment of the present invention, the port body 280 is connected to the implanted intracorporeal conduit 205 by pushing the conduit 205 into the segment 215 of the port body. In another embodiment of the present invention, the conduit 205 can be pushed over the segment 215 of the port body. In another embodiment of the present invention, the conduit 205 and the inner wall of the segment 215 are made with matching threads 208, such that conduit 205 can be threaded into the segment 215. In one embodiment of the present invention, the conduit 205 is pushed or threaded into the segment 215 up to a stop 220. The width of stop 220 matches the wall size of conduit 205 such that the inner lumen presents no obstacle to the flow through the conduit and laminar flow is ensured. After inserting the conduit 205 into or over segment 215 of the port body, a band or ring 210 is deployed to additionally secure in place the connection between the conduit 205 and the port body segment 215.

In one embodiment of the present invention, the inner lumen 282 contains valve 212. In one embodiment if the present invention, valve 212 is a 3-way valve, infusion, aspiration, and closed, 213. In another embodiment of the present invention, the valve 212 only opens when pushed by the tip of an access device, e.g., a syringe, when said access device is inserted into the lumen of said port.

Port body 280 and port segments 215, 222 and 230 can be made of any suitable, biocompatible material, such as stainless steel, titanium, alloys, silicone, polymers e.g., polyurethanes, PTFE, lightweight polysulphone or of any combination of the above.

In oner embodiment of the present invention, port 200 can be printed using multi-material 3D and 4D printing technologies.

Walls 222 and 230 of port 200 are shaped and made of a material and processed such as to ensure smooth liquid flow with minimal wall friction and minimal turbulence. The size of the angle 225 between the longitudinal axis of conduit 205 and the longitudinal axis of said port body can be chosen depending on the location of implantation of the port and of the preferred direction of the conduit 205, for example relative to skin surface. Further, angle 225 can be chosen as a function of the technique used (tunneled or not) and of the desired location of skin access.

In one embodiment of the present invention, port 200 is attached to the skin at the location of implantation using an auto-adhesive layer 260, for example using a hydrocolloid acrylic adhesive. In one embodiment of the present invention, the auto-adhesive provides antimicrobial protection with integrated chlorhexidine gluconate (CHG). After placement, port 200 can be additionally secured in place with sutures to the skin through the port holes 270 provided in the port body for this purpose. In one embodiment of the present invention, the sutures are absorbable or are removed once the self-adhesive layer 260 is safely secured to the skin.

In one embodiment of the present invention, implantable medical mesh 285 is attached to the body 280 of transcutaneous port 200. Such a mesh bag provides additional anchoring means adapted for tissue ingrowth, tissue encapsulation or tissue adhesion and ensures greater stability and additional protection of the implanted port. In another embodiment of the present invention, the exterior surface of port body 280 is made of such material and processed in such a way as to ensure the additional anchoring means through tissue integration mentioned above.

In one embodiment of the present invention, a protective cap 250 can be attached to body 280 of the transcutaneous port while the port is not in use. In one embodiment of the present invention, the cap is attached (screwed) and detached (unscrewed) by rotation 265 using the threads 290 in port body 280 and the matching threads 275 of cap 250. In one embodiment of the present invention, the connection at level 240 between cap 250 and body 280 is sealed using matching seals 245. The seal 245 can be made of different materials, e.g., rubber or silicone. Seal 245 can also be made of different polymers in a matching pair with the seal 235 inside the wall of the body 280. According to the present invention a matching male-female profile between the seal 245 and the seal 235 is preferably used that prevents fluid from flowing out of the conduit 282 when the cap is attached to the port body. In one embodiment of the present invention, such profile consists of concentric shapes on the seals 245 and 235 that allow the male portion of the profile situated on seal 245 to enter a groove on seal 235, while cap 250 is rotated for closure.

In one embodiment of the present invention, protective cap 250 has a safety mechanism to prevent accidental opening. In one embodiment of the present invention the safety mechanism is an anti-rotation lock 257. To unlock the cap, the lateral push-to-rotate 255 fixtures are used. Other anti-rotation locks can also be used: down-push and rotate, alignment of cap and body to a certain unique position, an external lock that needs to be pushed to release the cap for rotation, or an external lock or key of a shape that only matches a corresponding shape imprinted in the cap.

In one embodiment of the present invention, protective cap 250 is color coded. In one embodiment of the present invention, the surface 251 of the cap 250 can be red to indicated that the port is used to access an artery in the patient's body. In another embodiment, the cap surface 251 of the cap 250 can be blue to indicate that the port is used to access a vein in the patient's body.

In one embodiment of the present invention, protective cap 250 is sterile and single use.

In one embodiment of the present invention, protective cap 250 can be made of any suitable material such as silicone, rubber, or polymers (e.g., polyurethanes, PTFE) or a combination of the above. Stainless steel or alloys like nitinol can be used for the seal 245 or for providing any required reinforcements.

In one embodiment of the present invention, a patch or gel pad 298 is applied over cap and over the surrounding skin to provide additional barrier to microbes and external contaminants and to reduce port colonization and CRSBI (catheter related blood stream infections). In a preferred method of use, patch 298 is to be replaced with each use of the transcutaneous port together with cap 250.

In one embodiment of the present invention, transcutaneous port 200 provides variable blood flow characteristics in order to accommodate either arterial or venous flow. In one embodiment of the present invention, inner lumen 282 has a smaller diameter at its distal end compared to a larger diameter at its proximal end in order to increase the pressure of fluid infusion into conduit 205 or to decrease the pressure of fluid aspiration from conduit 205. In another embodiment of the present invention, inner lumen 282 has a larger diameter at its distal end compared to a smaller diameter at its proximal end in order to decrease the pressure of fluid infusion into conduit 205 or to increase the pressure of fluid aspiration from conduit 205.

In one embodiment of the present invention, transcutaneous port 200 can be detached from the conduit 205 and from the skin 202 and can be replaced with another port 200 or with any other port connectable to conduit 205.

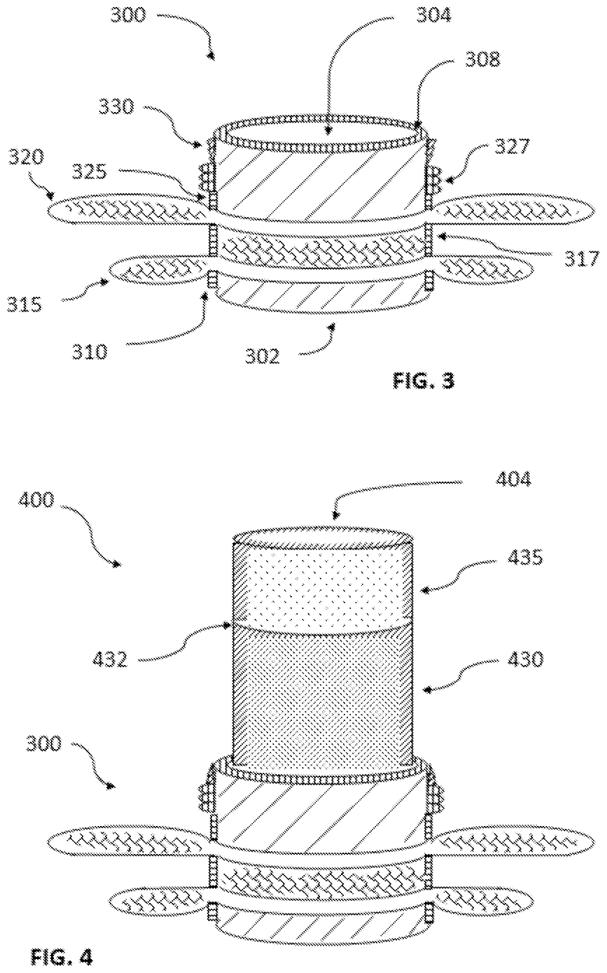

FIG. 3 illustrates an example embodiment of a tubular device anchor according to the present invention. Device 300 comprises a body 302 having an inner lumen 304, an expandable distal segment 315, an expandable waist segment 317, an expandable proximal segment 320, and attachment segments 327 and 330.

In one embodiment of the present invention, distal segment 315 self-expands in the lumen of a conduit, e.g. a blood vessel or graft and abuts the conduit wall from the inside of the conduit. In one embodiment of the present invention, proximal segment 320 self-expands outside the conduit and abuts said conduit wall from the outside of the conduit. In one embodiment of the present invention, waist segment 317 has a width approximately equal to the thickness of the blood vessel or graft wall, connects said distal 315 and proximal 320 segments of the device and ensures that the distal and proximal segments are applying the appropriate amount of pressure to said conduit wall as to keep the device steady on the conduit wall.

In one embodiment of the present invention, the proximal segment 320 is larger in size than the distal segment 315 in order to provide a larger footprint outside the blood vessel and a smaller footprint inside the blood vessel. The larger footprint outside the blood vessel provides better protection and more reliable attachment to the blood vessel. The smaller footprint inside the blood vessel reduces turbulences in flow and the risks associated with thrombosis and vessel wall damage, e.g., stenosis.

Distal segment 315 and proximal segment 320 of device 300 can be made of medical-grade stainless steel or of cobalt alloy or of a shape memory alloy, e.g., nitinol or of other shape memory materials, such as shape memory polymers, or of other superelastic material.

In one embodiment of the present invention, said distal and proximal segments, as well as the waist segment, are appropriately sized pieces of alloy fabric. One can solder, braze, weld or otherwise affix the ends of the braided alloy fabric to a distal ring 310, to waist 317, if the case, and to a proximal ring 325.

In one embodiment of the present invention, said distal and proximal segments are laser cut struts in a tube of shape memory material and pre-shaped or preprogrammed in the desired configuration as described further herein. In one embodiment of the present invention, said device is formed by laser cutting patterns in shape memory material tubing or sheets that are subsequently rolled into cylinders whose edges are welded together.

In one embodiment of this invention, the wall thickness 308 of the tubular device structure is in the range of 0.1 mm in order to allow for deployment in small peripheral blood vessels. In another embodiment of the present invention, each or all of the said segments of the device 300 or device 300 in its entirety can be printed using multi-material 3D and 4D printing technologies.

In one embodiment of the present invention, distal segment 315 is drug coated in order to minimize the damage to the vessel wall and prevent stenosis and thrombosis, e.g., with paclitaxel or sirolimus. In another embodiment of the present invention, said distal segment is gold plated in order to lower thrombogenicity.

In one embodiment of the present invention, attachment segment 327 is used to attach a delivery element to device 300 according to the present invention. In one embodiment of the present invention, a delivery element is attached to device 300 by screwing it onto the attachment threads 327 and is detached from the said device by unscrewing it. In another embodiment of the present invention, the attachment mechanism 327 can be a “bayonet” lock.

In one embodiment of the present invention, attachment segment 330 is used to attach device 300 to the distal end of a conduit, e.g., arteriovenous graft or arteriovenous shunt. In one embodiment of the present invention the conduit is attached to the device 300 by force pushing the end of the conduit over the attachment segment and securing it in place using heat shrink tubing. In another embodiment of the present invention, attachment mechanism 330 can be a “bayonet” lock or a threaded screw-unscrew attachment. In another embodiment of the present invention, attachment mechanism 330 can be made to match an appropriate segment of the graft or shunt end to which it is connected.

In one embodiment of the present invention, attachment segments 327 and 330 can be made of the same material as the any of the other segments of device 300 or of stainless steel, silicone, PTFE, polyurethane or of any other suitable biocompatible material.

In one embodiment of the present invention, device anchor 300 can have a circular cross-section. In another embodiment of the present invention, the device anchor 300 can have an elliptical cross-section, in which case the longer axis of such ellipsis must be aligned with the longitudinal axis of the target conduit.

In one embodiment of the present invention, the inner cross-section of the device 300 is of such size as to ensure the appropriate debit of fluid required for a hemodialysis procedure, e.g., in the range of 300-600 ml/sec.

In one embodiment of the present invention, device 300 or at least one of its segments is radiopaque and/or echogenic. In one embodiment of the present invention, device 300 is MRI compatible, power injectable and Latex, DEHP and PVC free. In one embodiment of the present invention, device 300 is marked such as to allow an easy deployment under ultrasound guidance and an easy alignment along the blood vessel in the case of an elliptical embodiment.

FIG. 4 illustrates one example embodiment of a device according to the present invention. Device 400 consists of a device anchor 300 and an intracorporeal conduit 404. In one embodiment of the present invention, conduit 404 extends into the anchor 300 to the distal end of said anchor such that the distal end of said conduit and the distal end of said anchor are flush. In one embodiment of the present invention, the distal end of intracorporeal conduit 404 is fixedly attached to the distal end of device anchor 300. In another embodiment of the present invention, the distal end of intracorporeal conduit 404 is fixedly attached to waist 317 of device anchor 300.

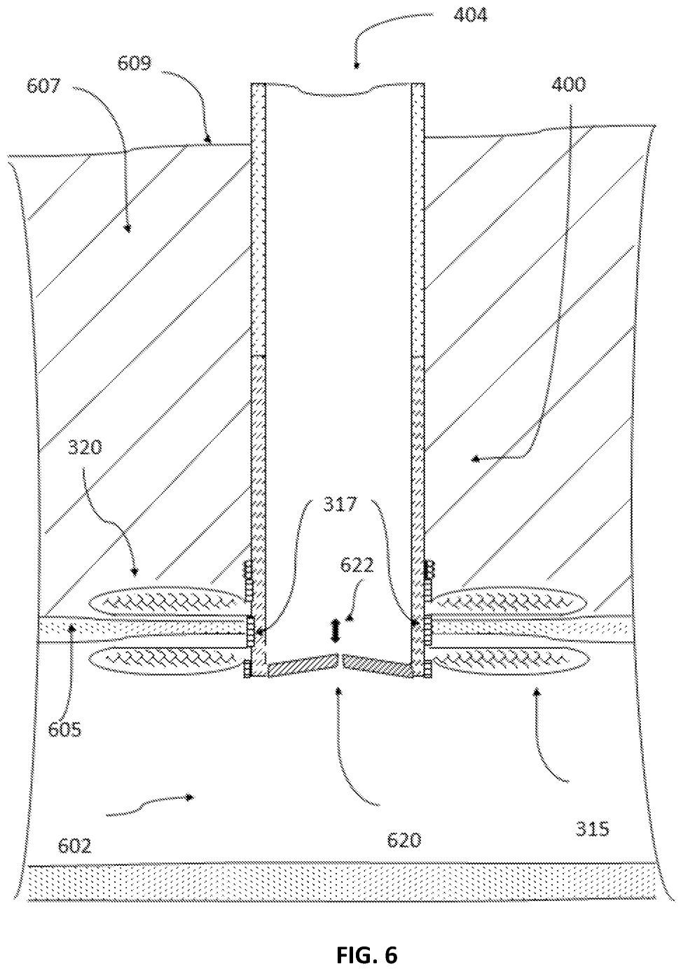

In one embodiment of the present invention, the conduit 404 comprises a 3-way (infusion, aspiration, and closed) valve, as illustrated at 620 on FIG. 6. The valve minimizes the risk of conduit blockage due to thrombosis by still allows for easy infusion and aspiration. In another embodiment of the present invention, the conduit 404 extends only partially into anchor 300 and a three-way valve is built into the distal end of said anchor 300 at the level of the distal ring 310. In one embodiment of the present invention, conduit 404 and valve 620 are made from the same material, e.g., silicone, PTFE, polyurethane. In one embodiment of the present invention said conduit and valve can be 3D or 4D printed.

In one embodiment of the present invention, the device can be built in different sizes, such as to allow deployment in larger vessel, e.g., in the range of 6 mm in diameter in the upper arm or upper legs and in smaller vessels, for example in blood vessels in the range of 2 mm diameter corresponding to arteries and veins of the lower arm or lower leg. Further, the sizes of the device are such as to allow deployment at deeper depths, e.g., in the range of 6 cm in the upper arm and leg and at shallower depths, for example in the range of 2 cm in the lower arm and leg.

In one embodiment of the present invention, after the percutaneous deployment and attachment of device 400 to the wall of a blood vessel or graft as further described herein, the proximal end of conduit 404 remains out of the patient's body such that it can be connected to another device, as described further herein. In one embodiment of the present invention the said conduit is trimmable and marked with cm markings.

In one embodiment of the present invention, conduit 404 is made of two segments 430 and 435 of different materials with different properties. In one embodiment of the present invention, segment 430 extends from the distal end of the conduit (and of anchor 300) for a minimal length equal to the length of anchor 300 when said anchor is in a compressed (non-expanded) configuration as illustrated and described in FIG. 6.

In one embodiment of the present invention, segment 430 is made of such material as to be more rigid than segment 435 in order to allow for the proximal segment of anchor 300 to easily slide over said more rigid segment 430 during the deployment of the device. In one embodiment of the present invention, segment 430 is made of such material as to allow for reliable attachment of anchor 300 to conduit segment 430 at their respective distal ends. In one embodiment of the present invention, segment 430 can be made of silicone, PTFE, polyurethane or similar materials of different rigidity than segment 435 and/or braided with braids made of stainless steel or nitinol or similar alloys. In one embodiment of the present invention, segment 430 is made of titanium in order to minimize blood clotting. In one embodiment of the present invention, segment 430 is gold plated in order to lower thrombogenicity. Segments 430 and 435 can be attached together using heat shrink tubing, overmolding or co-extrusion, by using an attachment mechanism 330, by pushing segment 435 over or into segment 430 or segment 430 can be made by additionally braiding and hardening segment 435.

In one embodiment of the present invention, the ratio between the outer diameter of said device anchor 300 in a compressed (non-expanded) configuration and the inner diameter of the more rigid distal segment 430 of conduit 404 is minimized by minimizing the wall thickness of said device anchor and the wall thickness of said more rigid distal segment such as to allow for fluid debits required for hemodialysis in the range of 300-600 ml/min while allowing attachment to small peripheral veins and arteries in the range of 2-3 mm in diameter.

In one embodiment of the present invention, segment 435 extends from the proximal end 432 of segment 430 to the proximal end of conduit 404 outside the patient's body. In one embodiment of the present invention, segment 435 is made of silicone, PTFE, polyurethane, or similar materials.

In one embodiment of the present invention, device 400 can be removed from the vessel wall and from the patient's body percutaneously through a procedural sheath according to the present invention. The opening left in the vessel wall after removal can be closed through the procedural sheath or over a guidewire using a standard vessel closure device. In one embodiment of the present invention, a method for percutaneously removing said device from the wall of a blood vessel comprises the following steps:
1. Detaching the proximal end of the intracorporeal conduit from the transcutaneous port, if attached.
2. Inserting a guidewire through the intracorporeal conduit into the target blood vessel.
3. Inserting a delivery element over the intracorporeal conduit until the distal end of the delivery element reaches the proximal end of the device anchor.
4. Inserting a delivery sheath over the delivery element until the delivery sheath reaches the outside of the blood vessel wall.
5. Under ultrasound imaging guidance, attaching the attaching mechanism of the delivery element to the attaching mechanism of the device anchor.
6. Pulling the delivery element thus pulling the device anchor and the distal end of the intracorporeal conduit into the delivery sheath while pushing the delivery sheath into the opening left in the wall of the blood vessel or graft by the device anchor.
7. After the delivery sheath has been pushed into the blood vessel or graft, pulling back the delivery element thus pulling back and out of the body the device anchor and the intracorporeal conduit through the delivery sheath.
8. Closing the wall opening in the wall of the blood vessel or graft through the delivery sheath and over the guidewire using a closure device.

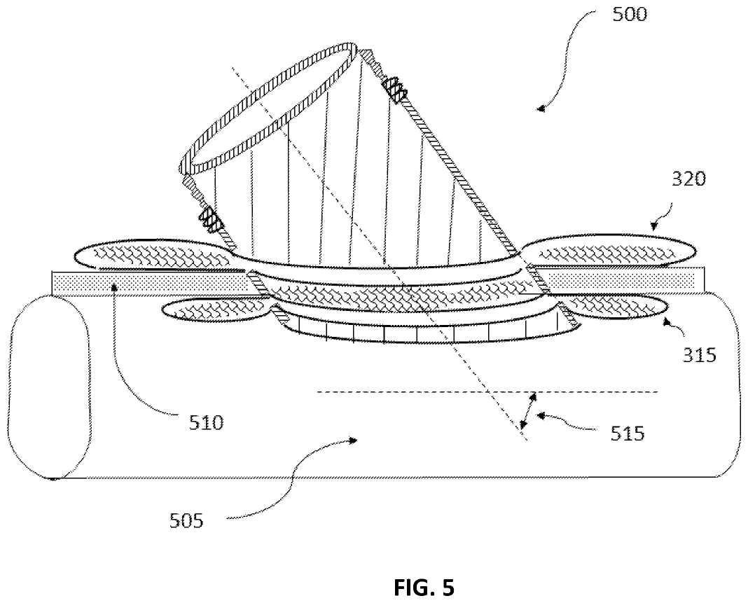

FIG. 5 illustrates an example embodiment of a device anchor 500 with a variable angle 515 between said anchor body and its expandable segments 315 and 320. Device anchor 500 can be attached to the wall 510 of a blood vessel or a graft 505 such that an appropriate value for angle 515 between the longitudinal axis of said blood vessel or graft 505 and the longitudinal axis of the inner lumen of said device 500 can be chosen in order to provide optimized fluid dynamics though the device relative to the location of the device attachment and to the desired path of attached intracorporeal conduit.

FIG. 6 illustrates an example embodiment of a device 400 deployed in a blood vessel or a graft 602 through patient skin 609 and surrounding tissue 607 according to the present invention. The distal segment 315 of device 400 is placed in the lumen 602 of the blood vessel, apposed from the inside to the inner surface of the wall 605 of the target conduit, e.g., a blood vessel. The proximal segment 320 of device 400 is placed outside the vessel, apposed from the outside to the outer surface of the wall 605 of said target conduit. Waist segment 317 of device 400 connects the distal and proximal segments of the device and ensures that the said segments are applying the right amount of the pressure to the vessel wall 605 as to keep the device steady in place. The conduit 404 of device 400 exits the patient skin and can be trimmed and attached to an external device.

In one embodiment of the present invention, the 3-way (622) (infusion, aspiration, and closed) valve 620 minimizes risk of conduit blockage due to thrombosis by allowing infusion and aspiration of fluid to and from target conduit 602 through device conduit 404 only upon demand and by preventing fluid, e.g., blood from entering conduit 404 when the device is not used for infusion or aspiration.

FIG. 7 illustrates a superimposed view 700 of another embodiment of an expanded (pre-shaped) and non-expanded device anchor 705 attached to the distal segment of intracorporeal conduit 710 comprising distal valve 740. A delivery element can be attached to and detached from said device anchor by using attaching and detaching mechanism 715. Proximal expandable segment 720 and distal expandable segment 730 are configured as expandable struts, wherein the struts width is optimized to apply ensure pressure on the target vessel wall and to minimize the device weight and contact surface with said vessel wall, and wherein the proximal segment 720 is larger in size than the distal segment 730 in order to ensure reliable attachment to the vessel wall while minimizing the amount of material present within the vessel, thus minimizing the risk of thrombosis and stenosis.

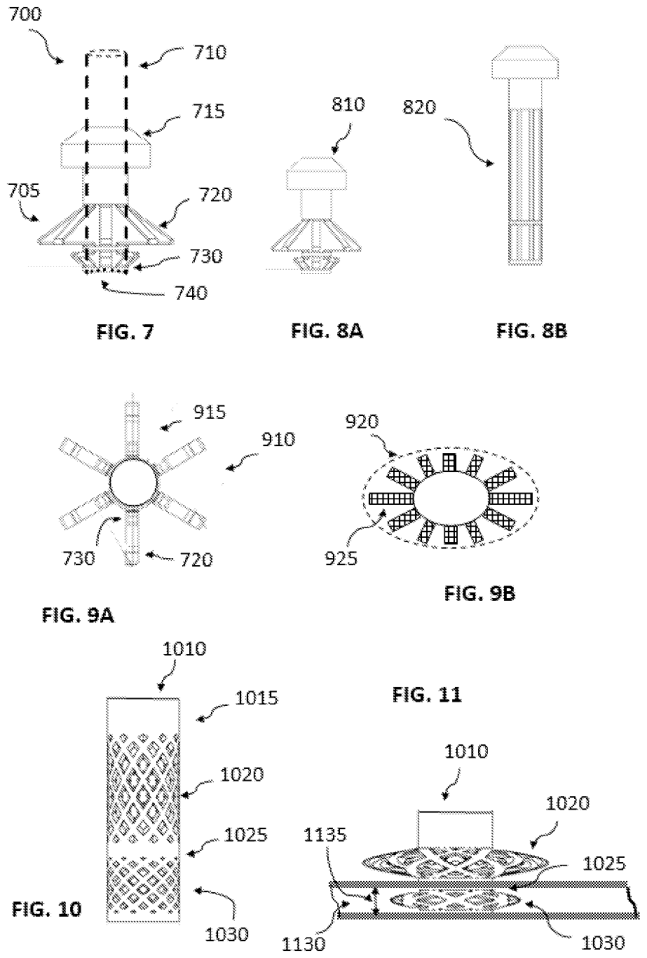

FIG. 8A illustrates a 3D view of device anchor 705 in an expanded (pre-shaped) configuration with struts cut from a tube of shape memory material 810 after shape setting of said material according to the present invention.

FIG. 8B illustrates a 3D view of device anchor 705 in a non-expanded configuration with struts cut from a tube of shape memory material 820 before shape setting of said material according to the present invention.

In one embodiment of the present invention, device anchor 705 is a thin walled cylindrical structure, wherein the distal, waist and proximal segments of said device are formed using a shape memory material, said distal and proximal segments consisting of several longitudinal struts disposed around the circumference of said cylindrical structure, said waist segment being ring-shaped, wherein said longitudinal struts of said proximal segment are attached at their distal end to said waist ring, wherein said longitudinal struts of said proximal segment are attached to a second ring at their proximal end, wherein said longitudinal struts of said distal segment are attached at their proximal end to said waist ring, wherein the distal and the proximal segments have the same or a different number of struts and, wherein the struts have the same or different widths, and wherein the struts are disposed equidistantly or at variable distance from each other around the circumference of said cylindrical structure 820.

In one embodiment of the present invention, said struts of said distal and proximal segments are pre-shaped through shape setting in their radially expanded and longitudinally compressed configuration, wherein each longitudinal struts of the proximal segment is bent outwardly as to be set in a V or U-shape, wherein the two ends of each of the V or U-shaped struts are attached to the proximal ring and to the waist ring respectively.

In one embodiment of the present invention, said struts of said proximal segment are coupled to each other by structures extending generally laterally.

FIG. 9A illustrates an example embodiment of a circular cross-sectional view 910 of an expanded distal end of device anchor 705 according to the present invention. The struts 915 are rectangular and disposed regularly in a circular pattern around a circular inner lumen. The struts of proximal segment 720 are of equal lengths. The struts of the distal segment 730 are of equal lengths. However, the struts of the proximal segment 720 are longer than the struts of the distal segment 730 in order to provide a larger surface for attachment to the vessel or graft wall.

FIG. 9B illustrates another example embodiment of an elliptical cross-sectional view 920 of the expanded distal end of a device anchor according to the present invention. The struts 925 are rectangular, of different lengths as to provide an elliptical cross-section and disposed around an inner lumen of elliptical cross-section for better alignment with the longitudinal axis of the target vessel and thus allowing deployment in smaller vessels.

FIG. 10 illustrates a frontal view of an example embodiment laser cut strut pattern of a device anchor 1010 in a non-expanded configuration before shape setting according to the present invention. A tube 1015 of shape memory and/or superelastic material is cut using pattern 1020 for the proximal segment and a different pattern 1030 for the distal segment of said device anchor. The waist segment 1025 separates the distal segment 1030 from the proximal segment 1020.

FIG. 11 illustrates a 3D configuration view of an example embodiment of the laser cut strut pattern of device 1010 in an expanded and deployed configuration according to the present invention. The expanded proximal segment 1020 is deployed outside the blood vessel or graft wall and the expanded distal segment 1030 is deployed inside the blood vessel or graft in such a way as to expand over the whole inner lumen of said blood vessel or graft with blood flowing through the struts of expanded distal segment 1030. The length 1135 of the expanded distal segment inside the blood vessel equals the inner diameter of blood vessel or graft 1130. The waist segment 1025 of approximately the height of the thickness of the vessel or graft wall attaches the deployed device 1010 to said vessel wall, such that expanded proximal segment 1020 and expanded distal segment 1030 apply sufficient pressure on the vessel wall to keep said device 1100 steady in place.

In one embodiment of the present invention, the distal segment of said device comprises a plurality of struts forming a net-like structure 1030 that, when pre-shaped, expands radially and compresses longitudinally.

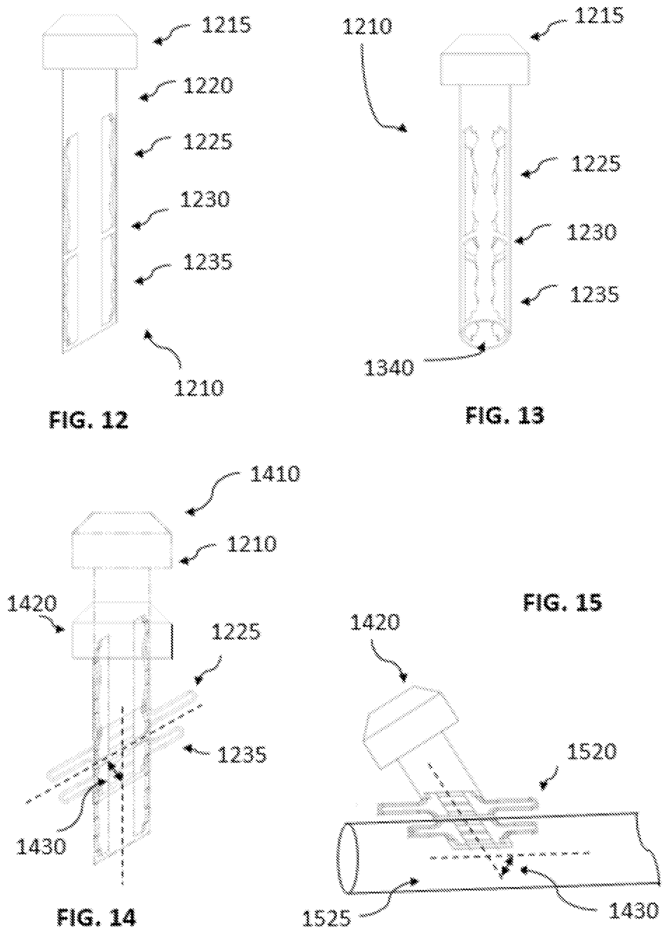

FIG. 12 illustrates a lateral view of yet another non-expanded example embodiment laser cut strut pattern of a device anchor 1210 suited for deployment at an angle with respect to the target vessel according to the present invention. A tube of shape memory and/or superelastic material 1220 is cut using certain patterns 1225 for the proximal segment, certain patterns 1230 for the waist segment, and certain patterns 1235 for the distal segment of a device anchor. The attaching mechanism 1215 can be cut from or welded to the same tube.

FIG. 13 illustrates a frontal view of device anchor 1210. The inner lumen 1340 of the device anchor is used to introduce a matching intracorporeal conduit and attaching the distal end of said conduit to the distal end or to the waist segment of said device anchor. The different laser cut patterns of proximal segment 1225, waist segment 1230, and distal segment 1235 allow to obtain different shapes and configurations of said device anchor through shape setting of the shape memory tube 1220. In one embodiment of the present invention, longitudinal struts of the distal and proximal segments are of variable width profiles that determine preferred bend locations of the struts for shape setting of the pre-shaped configuration of said device anchor. In one embodiment of the present invention, the plane of the waist ring 1230 is at a variable angle with respect to the cross-sectional plane of said cylindrical structure of said device anchor, and the plane of the distal end of the distal segment 1235 of said device anchor is parallel to the plane of the waist ring at said variable angle with respect to the cross-sectional plane of said device anchor

FIG. 14 illustrates a superimposed view 1410 of a non-expanded and expanded (pre-shaped) 1420 laser cut strut pattern of device anchor 1210 suited for deployment at angle 1430 with respect to the target vessel according to the present invention. In one embodiment of the present invention, segments 1225 and/or 1235 are double or multiple flipped.

FIG. 15 illustrates an example embodiment of device anchor 1210 deployed at angle 1430 with respect to the target vessel 1525 according to the present invention. The fixation distal and proximal segments 1520 are parallel to the vessel wall, while the longitudinal axis of the deployed device anchor 1420 are at angle 1430 with respect to the longitudinal axis of said target vessel 1525.

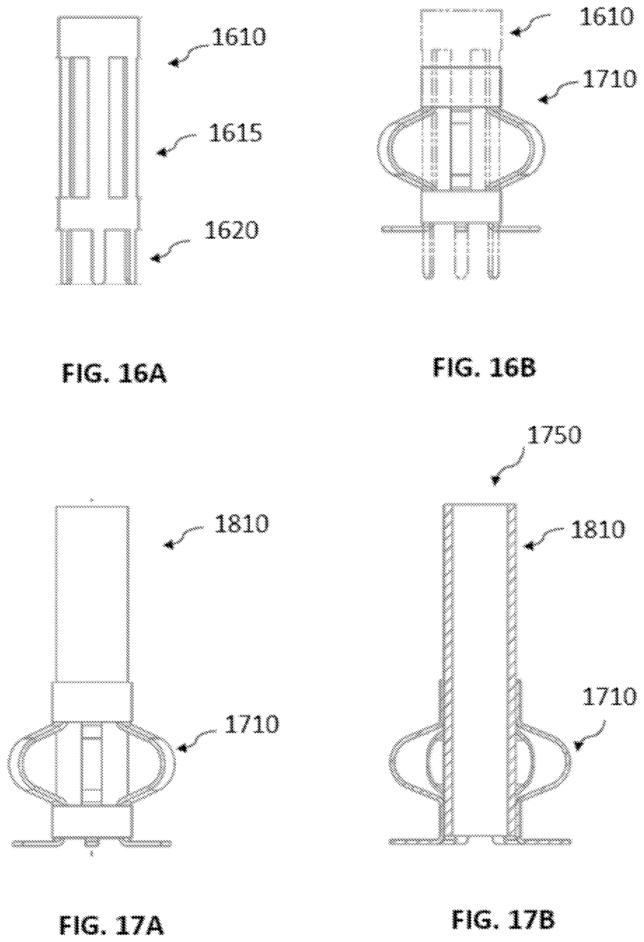

FIG. 16A illustrates a 3D view of another example embodiment laser cut strut pattern of a device anchor in a non-expanded configuration 1610 according to the present invention. The distal segment 1620 of said device anchor presents an open strut pattern that minimizes the volume of the material of the distal segment of the device anchor present in the blood vessel after deployment. In one embodiment of the present invention, proximal segment 1615 has a different number of struts than distal segment 1620. In one embodiment of the present invention, the struts of the proximal segment 1615 have a different width than the struts of the distal 1620. In one embodiment of the present invention, the struts of the distal segment 1620 have different lengths 925 in order to implement the elliptical shape 920 on FIG. 9B.

FIG. 16B illustrates a superimposed view of a non-expanded 1610 and expanded (pre-shaped) 1710 laser cut strut pattern of device anchor at FIG. 16A according to the present invention. When expanded, the longitudinal struts of the distal segment extend outwardly and perpendicularly to the longitudinal axis of the inner lumen of said device, as to minimize the contact surface between said struts and the inner vessel wall and the blood in the vessel and as to minimize the device weight.

FIG. 17A illustrates a 3D view of another example embodiment of an expanded (pre-shaped) device anchor 1710 attached to the distal segment 1810 of an intracorporeal conduit according to the present invention.

FIG. 17B illustrates a cross-sectional view 1750 of the expanded (pre-shaped) device anchor 1710 attached to the distal segment of the intracorporeal conduit 1810 as illustrated at FIG. 17A.

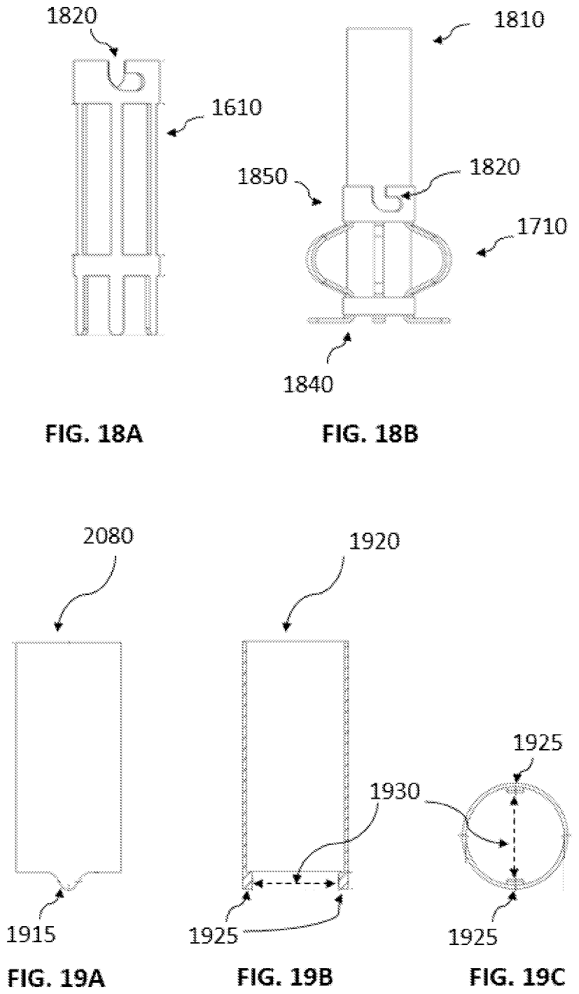

FIG. 18A illustrates a 3D view of an example embodiment laser cut strut pattern of a device anchor in a non-expanded configuration 1610 with a female-side bayonet attaching mechanism (bayonet mount or bayonet connector) 1820 according to the present invention.

FIG. 18B illustrates a 3D view of an example embodiment of the expanded (pre-shaped) device anchor 1710 with a female-side bayonet attaching mechanism (bayonet mount or bayonet connector) 1820, said device anchor being attached to the distal end 1840 of the intracorporeal conduit 1810 according to the present invention. The proximal end 1850 of the device anchor can slide over the distal segment 1810 of an intracorporeal conduit according to the present invention.

FIG. 19A illustrates a lateral view of an example embodiment of a tubular delivery element 2080 with a male-side bayonet attaching mechanism 1915 with male side pins oriented inwards according to the present invention. The male-side bayonet attaching mechanism 1915 matches the bayonet female-side attachment mechanism 1820 of the device anchor 1710.

FIG. 19B illustrates a longitudinal section 1920 of an example embodiment of a tubular delivery element 2080 with a male-side bayonet attaching mechanism 1915 according to the present invention. Pins 1925 of the male-side bayonet mechanism are interior to the tubular delivery element 2080 such that the distance 1930 between the inner edges of the bayonet pins is equal or slightly larger than the outer diameter of distal segment 1810 of the intracorporeal conduit such as to reliably engage the matching bayonet attaching mechanism 1820 of said device anchor and to slide over the distal segment of the intracorporeal conduit 1810. After attaching the bayonet connector 1915 of the delivery element, i.e., its respective pins 1925 to the corresponding bayonet connector 1820 of the device anchor 1710, the delivery element 2080 can be used to pull the proximal end of the device anchor 1710 along the distal segment of the intracorporeal conduit 1810 as described further herein.

FIG. 19C illustrates a cross-sectional view of an example embodiment of a tubular delivery element with a bayonet attaching mechanism according to the present invention, whereby the pins 1925 of the bayonet connector are internal and oriented inwards to the delivery element in order to be able to attach to the matching bayonet connector 1820.

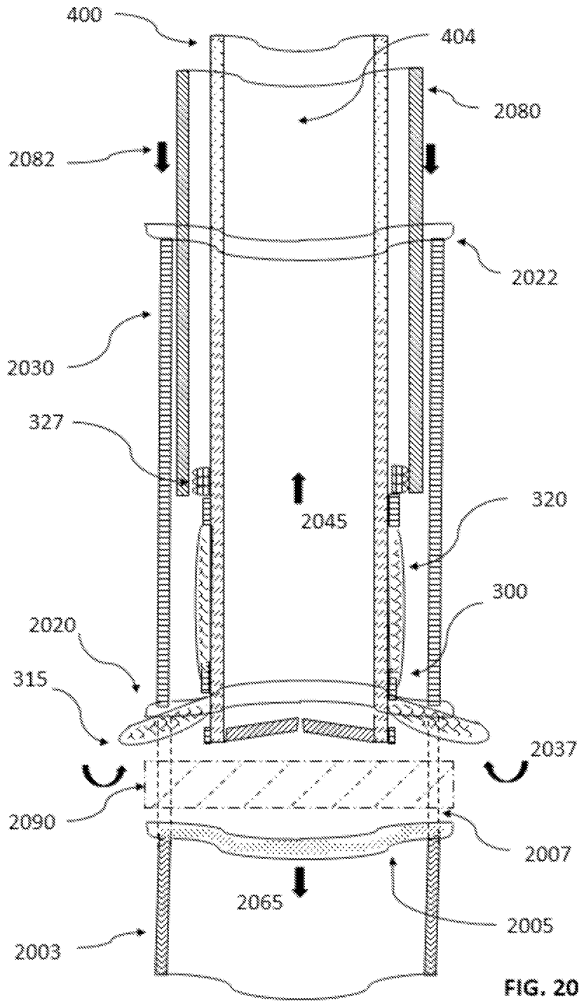

FIG. 20 illustrates an example embodiment of a method of introducing device 400 into delivery sheath 2003 by using loader 2030 and delivery element 2080 according to the present invention. In one embodiment of the present invention, loader 2030 is a tubular structure with female luer connectors at both its distal 2020 and proximal ends 2022. At the distal end, female luer connector 2020 of loader 2030 can be connected to female luer connector 2005 of delivery sheath 2003 using a male-to-male luer connector 2090. The connection is realized such as to ensure alignment 2007 between the inner wall of the loader 2030 and the inner wall of the lumen of the delivery sheath 2003.

In one embodiment of the present invention, the delivery sheath 2003 can be connected at its proximal end to a hemostasis valve 2170. In such case, the male-to-male luer connector 2090 connects the female luer connector 2020 of loader 2030 to the female luer connector of hemostasis valve 2170 and device 400 is transferred from loader 2030 to delivery sheath 2003 through the connector 2090 and through the hemostasis valve.

In one embodiment of the present invention, delivery element 2080 is a tubular structure, with an attachment mechanism at its distal end that can be attached to the matching attaching mechanism 327 of device 400. The delivery element is made of such materials as to be flexible enough and to provide enough torque and push/pull strength in order to allow for the use described herein.

The loader 2030, delivery element 2080, and the delivery sheath 2003 can be made of materials such as silicone, PTFE, polyurethane or other similar materials. In a preferred embodiment according to the present invention, loader 2030 is transparent in order to be able to visualize that device 400 is properly loaded.

In one embodiment of the present invention, loader 2030, delivery element 2080, and/or delivery sheath 2003 can be made using 3D and 4D printing of appropriate biocompatible materials.

In one embodiment of the present invention, a method of loading the device 400 into loader 2030 and deploying it in a blood vessel through a delivery sheath 2003 consists of the following steps:
1. The distal end of delivery element 2080 is inserted into a peel-away loader 2030 at the loader's proximal end and is pushed towards the loader's distal end such that the distal end of the said delivery element exits the distal end of said loader. The proximal end of said delivery element remains outside the proximal end of said loader.
2. The proximal end of intracorporeal conduit 404 is passed through delivery element 2080 (that has previously been inserted into the loader 2030) starting at the distal end of the said delivery element, such that device anchor 300 of device 400 remains outside the distal end of the said delivery element 2080. The proximal end of said intracorporeal conduit 404 exits the delivery element through the proximal end of said delivery element.
3. The distal end of the delivery element 2080 is attached to device anchor 300 using the attaching mechanism 327.
4. Device 400 is pulled back (2045) into the loader 2030 by pulling back the delivery element 2080 such that the device anchor 300 is pulled back and compressed in the inner lumen of loader 2030. FIG. 20 illustrates an intermediate step of device loading according to the present invention wherein the proximal segment 320 of device anchor 300 of device 400 is compressed inside the loader while the distal segment 315 of the device anchor 300 of device 400 is in the process of flipping (2037) and being compressed into the loader.
5. After device 400 is fully loaded into loader 2030 and after the delivery sheath 2003 has been properly placed in the patient's body in the target vessel, the distal end of said loader is connected to said delivery sheath using a female luer lock to female luer lock connector 2090.
6. Device 400 is then fully pushed (2065, 2082) into the delivery sheath 2003 by pushing the delivery element 2080 and, after the distal end of device 400 has been completely inserted in said delivery sheath, the loader 2030 is disconnected and pulled back or peeled away and removed.
7. Device 400 is placed in the vessel wall using the delivery sheath 2003 and the delivery element 2080 by a combination of pushing said delivery element and pulling back said delivery sheath.
8. After verification by ultrasound imaging of proper placement of device 400 in the vessel wall, delivery element 2080 is detached from the attaching mechanism 327 and pulled back out of the delivery sheath 2003 or peeled away and removed.
9. Finally, the delivery sheath 2003 is pulled back or peeled away and removed.
10. Through the entire deployment procedure, a hemostasis valve 2170 with extension tube 2175 and stopcock 2180 can be connected to the female luer lock 2005 of the delivery sheath 2003 and can be used as required for flushing the delivery system and controlling back-bleeding.

In another embodiment of the present invention, steps 5, 6, and 7 of the above described method of loading the device 400 into loader 2030 and deploying it in a blood vessel through a delivery sheath 2003 are replaced by the following steps:
1. After device 400 is fully loaded into loader 2030 and after the delivery sheath 2003 has been properly placed in the patient's body in the target vessel, the distal end of said loader is pushed into the delivery sheath by pushing the proximal end of said loader until the distal end of the loader, which is flush with the distal end of the intracorporeal conduit, has reached the distal end of the delivery sheath.
2. Attaching the intracorporeal conduit of claim 1 to the vessel wall by allowing the device anchor of claim 1 to self-expand by a combination of pushing said delivery element and pulling back said loader.
3. Pulling back and/or peeling away the loader.

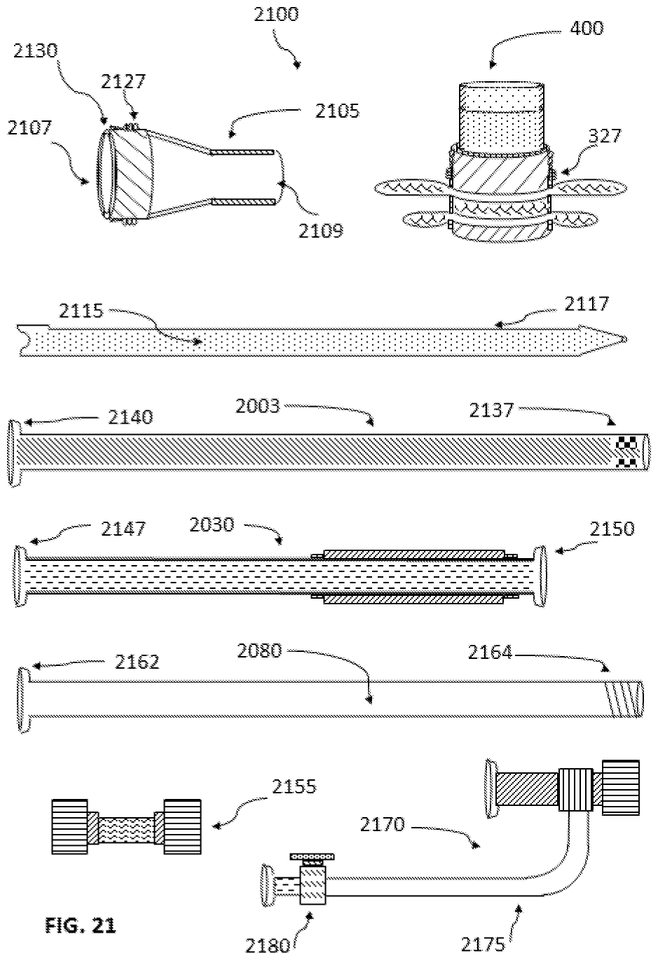

FIG. 21 illustrates an example embodiment of a deployment kit for percutaneous and sutureless deployment of device 400 according to the present invention. In one embodiment of the present invention, the kit 2100 consists of:
1. Implantable device 400.
2. Device-graft adapter 2105 used to adapt the size and/or shape of device 400 to a potentially different size and/or shape of a connected graft or shunt. Device 400 is connected at the distal end 2109 of adaptor 2105, while the graft or shunt is connected to the proximal end 2107 of adaptor 2105. Adaptor 2105 comprises an attachment segment 2127 for attaching a delivery element and segment 2130 for attaching said adaptor to a graft or shunt. Adaptor 2105 can be made of silicone, PTFE, polyurethane, nitinol, titanium or other similar materials and can be overmolded and/or co-extruded using a combination of such materials.
3. Dilator/introducer 2115 with a blood flow back path 2117 for accurate positioning of delivery sheath 2003 at the blood vessel wall as described herein and wherein said dilator/introducer can be deployed into the target vessel over a procedural guidewire.
4. Peel-away delivery sheath 2003 with a female luer connector 2140 at the proximal end. In one embodiment of the present invention, delivery sheath 2003 has echogenic orientation markers 2137 at the distal end for identification of the long axis of an elliptical cross-section and orientation along the longitudinal axis of the targe blood vessel or graft using ultrasound imaging.
5. Loader 2030 for loading device 400 and compressing device anchor 300 into a non-expanded configuration. In one embodiment of the present invention, loader 2030 is a tubular element comprising female luer connectors 2147 and 2150 at both its proximal and distal ends and is used for loading device 400 into delivery sheath 2003 as illustrated at FIG. 20. In one embodiment of the present invention, loader 2030 is peel-away. In one embodiment of the present invention, loader 2030 has echogenic orientation markers at the distal end for identification of the long axis of an elliptical cross-section and orientation along the longitudinal axis of the targe blood vessel or graft using ultrasound imaging.
6. In one embodiment of the present invention, delivery element 2080 is a tubular element comprising a female luer connector 2162 at its proximal end and a matching attachment mechanism 2164 at its distal end. Delivery element 2080 can be attached to matching attachment mechanism 327 of device 400 and thus loaded into loader 2030. In one embodiment of the present invention, delivery element 2080 is a peel-away tubular element such that, after deployment of device 400, delivery element 2080 is peeled away for removal. In one embodiment of the present invention, delivery element 2080 comprises echogenic orientation markers at the distal end 2164 for identification of the long axis of an elliptical cross-section and orientation along the longitudinal axis of the targe blood vessel or graft using ultrasound imaging.
7. Luer male-to-male connector 2155 for connecting loader 2030 to delivery sheath 2003.
8. Hemostasis valve 2170 with extension tube 2175 and stopcock 2180 to allow for flushing the delivery system and controlling back-bleeding as illustrated at FIG. 20 and described herein.

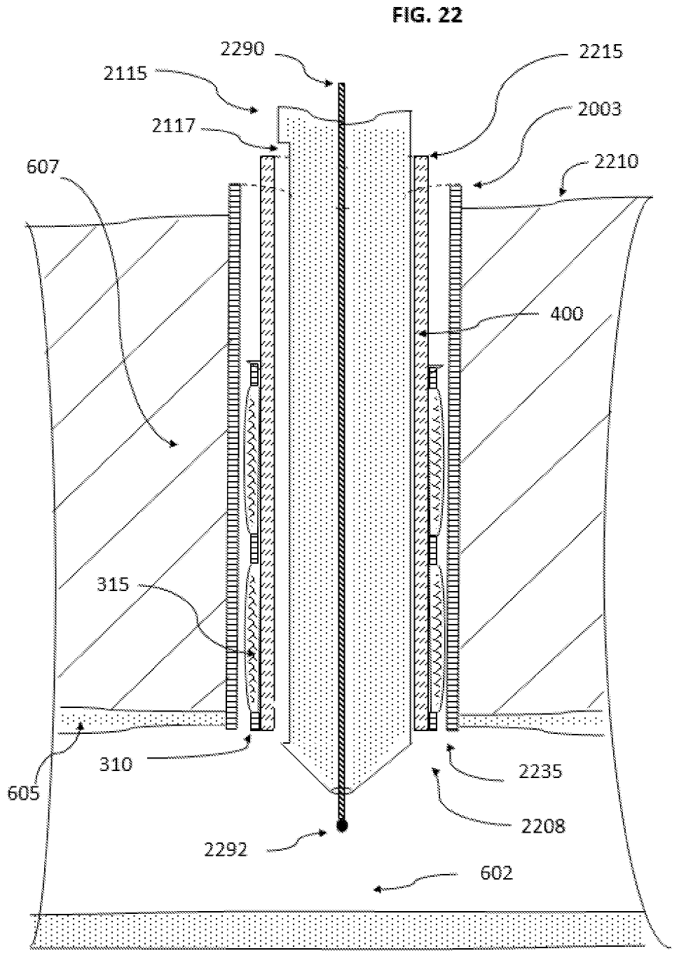

FIG. 22 illustrates an example embodiment of a method for percutaneous and sutureless deployment of device 400 over a guidewire through a procedural sheath according to the present invention. At FIG. 22, device 400 has been pushed into the delivery sheath 2003 such that the distal tip 310 of the device is flush with the distal tip 2235 of said delivery sheath. In one embodiment of the present invention, device 2215 does not contain a valve.

In order to gain access to the target blood vessel, a guidewire 2290 with an atraumatic tip 2292 has been introduced in the blood vessel 602 through an insertion needle penetrating the tissue 607 and the vessel wall 605. The insertion needle was retracted to leave distal end 2292 of guidewire 2290 in the blood vessel while the proximal end of guidewire 2290 remained outside the patient's skin 2210. Dilator/introducer 2115 was introduced in the blood vessel over the guide wire 2290 in order to enlarge hole 2208 in the blood vessel wall 605 and to facilitate the introducing of delivery sheath 2003 containing device 400.

In one embodiment of the present invention, in order to access blood vessels of small size, e.g., 2 mm in diameter, distal end 2235 of delivery sheath 2003 and the distal end 310 of device 400 need to be placed flush and flush with the inner wall of blood vessel 605. Thus, the space required for the deployment of the distal anchor segment 315 can be maximized. Flush alignment is verified during the placement procedure using the blood flow back channel 2117 present in the wall of the dilator/introducer 2115. To flush the distal tip of the device with the blood vessel wall, the ensemble consisting of procedural sheath 2003, device 400, and dilator/introducer 2115 is slowly advanced along the guide wire 2290 until control blood flow comes out through blood flow back channel 2117 and is visible at the proximal end of said channel 2117 outside the patient's skin. The correct alignment of the device at the vessel wall can be checked by ultrasound imaging using the echogenic markers of delivery sheath 2003 and/or of device 400.

In one embodiment of the present invention, in order to maximize blood flow through the device, the deployment of devices of larger sizes than the inner diameter of the targeted blood vessel is achieved by using device having an elliptic cross-section. In such a case, the long axis of the device's elliptical cross-section must be aligned with along the blood vessel, i.e., aligned with the longitudinal axis of the blood vessel. Enhanced ultrasound reflective markers and/or radiopaque markers integrated on the device 400 and/or on delivery sheath 2003 can be used to allow for longitudinal alignment using ultrasound and/or X-ray imaging guidance. Furthermore, a standard dilation of the target blood vessel can be performed if necessary during device deployment to enhance maneuverability of the device.

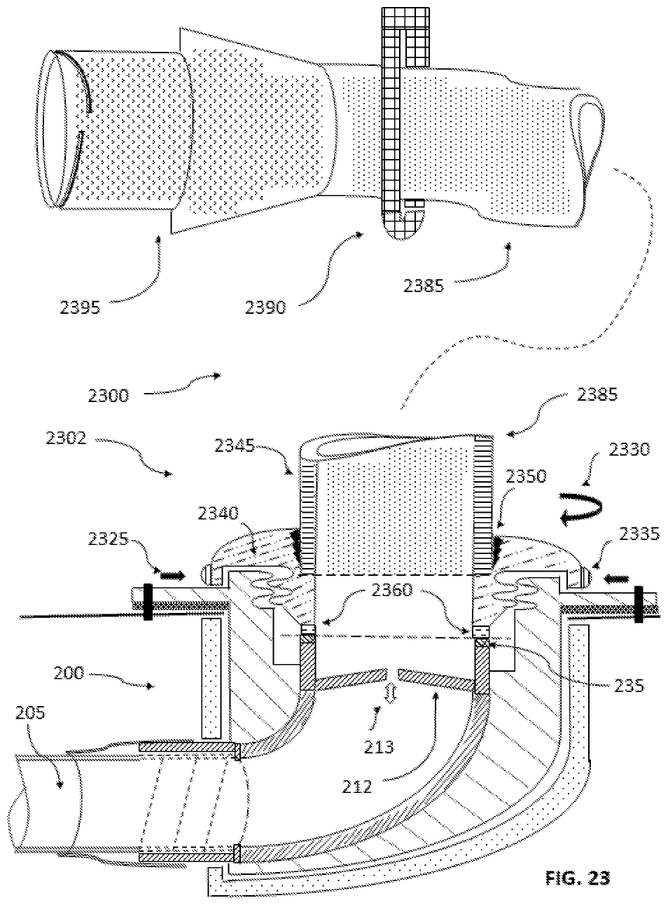

FIG. 23 illustrates an example embodiment of a device and method for needlelessly connecting a patient to a dialyzer according to the present invention wherein an extracorporeal connector 2300 is attached to a transcutaneous port 200. In one embodiment of the present invention, the extracorporeal connector 2300 consists of a body 2302 that can be attached to transcutaneous port 200, of a conduit 2385, and of a standard female Luer connector 2395 with a clamp 2390. The female Luer connector 2395 can be connected to an external device, e.g., a syringe or a hemodialysis machine. The clamp 2390 can be used to stop the fluid flow through the conduit 2385. When connected to a syringe, blood can be drawn from, and fluids can be delivered to the blood vessel connected to intracorporeal conduit 205 through port 200.

In one embodiment of a hemodialysis method according to the present invention, a first intracorporeal conduit 205 connected to a first port 200 is anastomosed to a vein and a second intracorporeal conduit 205 connected to a second port 200 is anastomosed to an artery. A first extracorporeal connector 2300 connected to the first venous access port 200 is connected to the venous line of a dialyzer and a second extracorporeal connector 2300 connected to the second arterial access port 200 is connected to the arterial line of a dialyzer. In one embodiment of the present invention, one or more elements of the connectors 2300 are color coded in blue as to identify venous line access and connection and in red as to identify arterial line access and connection. When the transcutaneous port 200 is in use, i.e., when connected to an extracorporeal connector 2300, the patch 298 and the cap 250 have been removed and replaced by the body 2302 of the extracorporeal connector 2300.

The conduit 2385 is connected to the element 2340 of the connector body 2302 through the connecting segment 2350. Depending on the materials of the conduit wall 2345 and of the body element 2340, connecting segment 2350 can be made using different technologies and materials, e.g., stainless steel, titanium, alloys, silicone, polymers e.g., polyurethanes, PTFE, lightweight polysulphone or of any combination of the above. In one embodiment of the present invention, conduit 2385 is glued to connector body element 2340.

In one embodiment of the present invention, element 2340 of the connector body is attached and removed to and from port 200 by rotating (2330) and pushing (2325) a push-rotate locking mechanism 2335. The safety mechanism 2335 that prevents accidental disengagement of the element 2340 matches the safety mechanism of port 200. In one embodiment of the present invention, safety mechanism 2335 is lateral-push-to-rotate anti-rotation lock. Other anti-rotation locks can also be used: down-push and rotate, alignment of cap and body to a certain unique position, or an external lock that needs to be pushed to release the cap for rotation.

The extracorporeal connector 2300 can be made of any suitable material such as silicone or polymers (e.g., polyurethanes, PTFE) or a combination of the above. Stainless steel or alloys like nitinol can be used for braiding the conduit 2385, for the seals 2360 and 235 or for providing any required reinforcements. Rubber or lightweight polysulphone can be used for the element 2340 of the body 200.

In one embodiment of the present invention, valve 212 is a 3-way valve 213 (closed, infusion, aspiration) that is normally closed and opens only under the negative pressure (aspiration) or positive pressure (infusion) generated by the device or dialyzer attached to connector 2395.

In one embodiment of the present invention, valve 212 is normally closed and opens only when the distal end of the extracorporeal connector 2300 is attached to the connector body 2302 such that the distal end of said connector pushes through valve 212 and opens it.

In one embodiment of the present invention, the extracorporeal connector 2300 is sterile and single use.

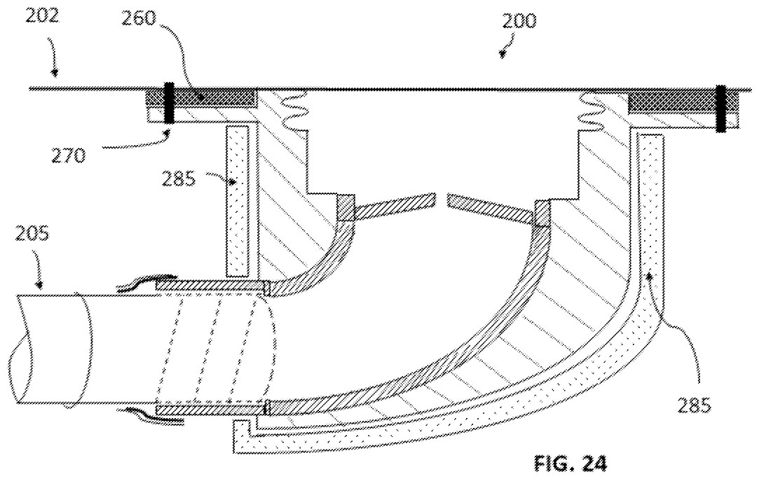

FIG. 24 illustrates another example embodiment of transcutaneous access port 200 placed subcutaneously under the patient's skin 202 and connected to intracorporeal conduit 205 according to the present invention. When said transcutaneous access port 200 is placed subcutaneously, the patient's skin covers said access port and protection patch 298 and protective cap 250 at FIG. 2 are no longer needed. Port 200 can be attached to the patient's skin using sutures 270 or adhesives 260 and/or fixed subcutaneously in the patient's body with an appropriate layer of medical mesh 285.

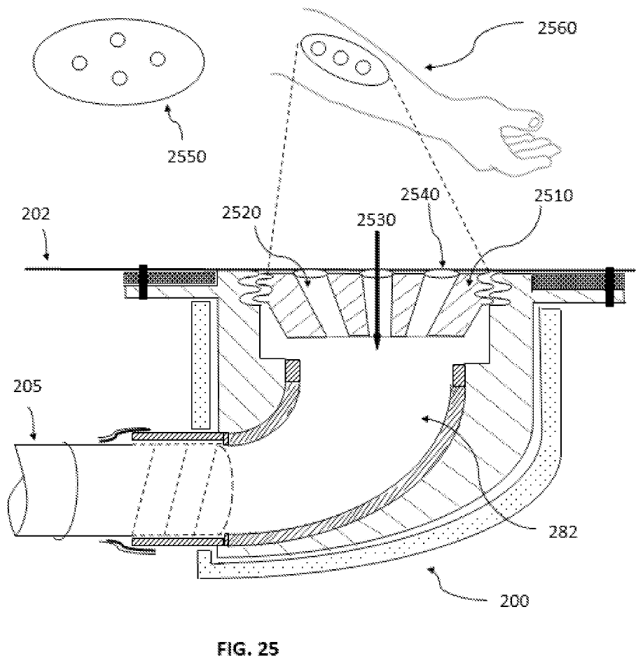

FIG. 25 illustrates another example embodiment of transcutaneous access port 200 placed subcutaneously and comprising an adaptor 2510 having one or several predefined needle access points or channels or buttonholes 2520 to provide one or more fluid paths to intracorporeal conduit 205 through the port's inner lumen 282 according to the present invention. Adaptor 2510 can be attached to (screwed into) port 200 prior to implantation and can be removed by unscrewing it through a skin incision. Each access channel 2520 has a more prominent proximal ending 2540 that can be sensed through patient's skin 202 to identify the point of needle insertion. By inserting a needle through the skin into a channel 2520, fluid can be infused or aspirated through said channel to and from the inner lumen 282 of port 200 and thus, to and from the intracorporeal conduit 205. In another embodiment of the present invention, channel 2520 is used to guide 2530 a needle or another type of the access device from the skin 202 into the main lumen 282 of said port 200.

In one embodiment of the present invention, adaptor 2510 is provided in different configurations as illustrated by 2550 and 2560 with channels or buttonholes 2520 having different locations for each configuration such that, when a different configuration is used, the access points on skin for needle puncture change locations. Thus, the skin is not punctured in the same location each time giving the skin time to heal from one puncture to the next, e.g., from one hemodialysis session to the next. Furthermore, each configuration of adaptor 2510 can have one or several access channels and an appropriate size corresponding to the number and location of the access channels.

In one embodiment of the present invention, adaptor 2510 is made of any suitable, biocompatible material, such as stainless steel, titanium, alloys, silicone, polymers e.g., polyurethanes, PTFE, lightweight polysulphone or of any combination of the above. For example, the body of adaptor 2510 can be made of polymers overmolded over channels 2520 made of stainless steel, titanium, or nitinol.

In the foregoing detailed description, various individual features are grouped together in order to streamline the disclosure. However, inventive subject matter also lies in such individual features and not only in their grouping together as disclosed in the foregoing embodiments. Thus, the following claims are hereby incorporated into the detailed description, with each claim standing on its own as a separate preferred embodiment. Therefore, the spirit and scope of the appended claims should not be limited to the description of the preferred versions contained herein.

1-8. (canceled)

9. A transcutaneous access port for hemodialysis (200) comprising a port body (280) with a subcutaneous segment (215) attachable to an intracorporeal conduit, said transcutaneous access port having an inner lumen profile (282), characterized in that the said inner lumen has tapering walls with a larger diameter at one end of said inner lumen and a smaller diameter at the other end of said inner lumen and a smooth transition between the larger and the smaller ends of said inner lumen such as to provide variable blood flow characteristics adapted to either arterial or venous blood flow while minimizing blood flow turbulences.

10. The transcutaneous access port according to claim 9, characterized in that the said transcutaneous access port comprises a valve (212).

11. The transcutaneous access port according to claim 10, characterized in that the said valve is configured to open when negative pressure or positive pressure is applied through the inner lumen of said port.

12. The transcutaneous access port according to claim 10, characterized in that the said valve is configured to open when a device is pushed into said port body through said valve.

13. The transcutaneous access port according to claim 9, characterized in that the said transcutaneous access port is configured to be implanted subcutaneously under a patient's skin (FIG. 24).

14. The transcutaneous access port according to claim 51, characterized in that the said access channels of said removable adaptor and their corresponding access points under the skin are provided in different configurations at different locations under the skin (2550) in order for needle punctures to be done at different locations on the skin as to allow the skin to heal from one needle puncture to the next.

15. The transcutaneous access port according to claim 9, characterized in that the said access port comprises an extracorporeal protection cap (250).

16. The transcutaneous access port according to claim 15, characterized in that the said cap is color-coded to allow for easy identification of blood vessel connections.

17. The transcutaneous access port according to claim 15, characterized in that the said cap comprises a safety mechanism (255) to prevent accidental opening and a ring-shaped seal (245) for fluid tight connection to the body of said access port.

18. The transcutaneous access port according to claim 9, characterized in that the inner diameter of the inner lumen (282) is smaller at its distal end compared to a larger diameter at its proximal end in order to increase the pressure of the fluid infusion through the connected intracorporeal conduit and whereby the transition between the smaller and the larger diameters is smooth in order to minimize fluid flow turbulences.

19. The transcutaneous access port according to claim 9, characterized in that the inner diameter of the inner lumen (282) is larger at its distal end compared to a smaller diameter at its proximal end in order to decrease the pressure of the fluid infusion through the connected intracorporeal conduit and whereby the transition between the smaller and the larger diameters is smooth in order to minimize fluid flow turbulences.

20. The transcutaneous access port according to claim 9, characterized in that the implantable medical mesh (285) is attached to the outside wall of said device, said mesh providing additional anchoring means adapted for tissue ingrowth, tissue encapsulation or tissue adhesion and ensures greater stability and additional protection of said implanted device.

21. (canceled)

22. The transcutaneous access port according to claim 9, characterized in that the said transcutaneous access port comprises an extracorporeal connector (2300) to provide needleless connection to a dialyzer, said extracorporeal connector comprising a conduit (2385) with a proximal end and a distal end, wherein said distal end includes a matching connector (2302) that can be attached to the device of claim 9 and said proximal end (2395) can be attached to a dialyzer.

23-49. (canceled)

50. The transcutaneous access port according to claim 9, characterized in that the longitudinal axis of the inner lumen of said subcutaneous segment and the longitudinal axis of the inner lumen of said port body can be configured at a variable angle (225) with respect to each other in order to adapt to the anatomy of the access and implant locations.

51. The transcutaneous access port according to claim 13, characterized in that the said transcutaneous access port comprises a removable adaptor (2510) with one or more access channels (2520) that can guide an access needle through the skin and into the inner lumen of said port (2530).

52. The transcutaneous access port according to claim 51, characterized in that the access channels (2520) of removable adaptor (2510) that are covered by the skin (202), can be located by an operator through the skin (2540).
